(19) United States
(12) Patent Application Publication
(10) Pub. No.: US 2005/0267432 A1
(43) Pub. Date: Dec. 1, 2005

(54) Disposable pad and disposing method and apparatus

(75) Inventors: Anders Karl Sundberg, Elwood (AU); Noel D. Ryan, Victoria (AU)

Correspondence Address: VOLPE AND KOENIG, P.C., UNITED PLAZA, SUITE 1600, 30 SOUTH 17TH STREET, PHILADELPHIA, PA, 19103, US

(73) Assignee: The Australian Invention Company, Melbourne Victoria (AU)

(21) Appl. No.: 10/522,971

(22) PCT Filed: Feb. 27, 2003
(86) PCT No.: PCT/AU03/00240

§ 371 Date: Jan. 31, 2005

(30) Foreign Application Priority Data
Jul. 31, 2002 (AU) 2002950507

(51) Int. Cl. A61F 13/15

(52) U.S. Cl. 604/385.13

(57) ABSTRACT

A nappy, diaper or sanitary pad assembly has an integral pouch made of a flexible fluid impermeable material in an elongated shape aligned inline lengthways with the pad at the outside bottom of the pad. The pouch contains a sealable pre formed bag within an inner portion and a pre-moistened towelletes in an outer portion. The two portions are separated by a flap.

### FIELD OF INVENTION

This invention relates to improvements in disposable pads and particularly to disposable pads in the form of nappies/diapers or sanitary/incontinence pads.

### BACKGROUND

Disposable nappies/diapers have been available for many years. However the convenience of facilities is lessening and likelihood of finding assistance for care of babies, incontinent people and menstruating women is less available in the public arena. It is therefore important to be more self sufficient in a discreet manner.

It is known to have a product attached to a disposable nappy/diaper, however such is usually merely adhesively connected. It is however not discreetly or very advantageously included but results in an ineffective bulkier product.

It is therefore an object of the invention to provide an improved nappy/diaper or sanitary/incontinence pad which overcomes the problems of the prior art by providing a discreet product which effectively provides a disposal system.

### SUMMARY OF THE INVENTION

In one form of the invention there is provided a disposable pad having an elongated absorbent pad element having a front side for contact with a body for receiving human excrements and an opposing rear side forming a pad backing; a backing strip extending over at least part of the pad backing and detachably attached to the disposable pad; and a disposal bag mounted between the backing strip and the pad backing wherein the disposal bag is unobtrusively in place when the disposable pad is in use and readily accessible for enclosing the disposable bag after use for hygienic disposal.

The flap strip can extend along the length of the nappy over a bag structure where the flap strip forms part of the bag cuts down on material used. It also results in being longer than bag structure so acts as a readily handled tag. This allows activation of the bag without separate handling. Further it covers the internal structure of the bag and stored products and provides a continuous outer surface to minimise ridges.

The invention also provides a disposable pad having an elongated absorbent pad element having a front side for contact with a body for receiving human excrements and an opposing rear side forming a pad backing and a double action backing strip formed by a first part attached to the disposable pad forming a storage pouch holding a disposal bag and a second part separately attached to the disposable pad able to hold other hygienic products and wherein the storage pouch is opened by pulling on the backing strip to first open the second part and release the further hygienic products and by further pulling the bag structure is able to enclose the used disposable pad for hygienic disposal.

This double action flap strip where a storage pouch is opened by pulling on the flap strip and by further pulling the bag structure is activated; this allows one handed operation and activates the bag without separate handling

Also according to the invention there is provided a disposable pad having an elongated absorbent pad element having a front side for contact with a body for receiving human excrements and an opposing rear side forming a pad backing, a backing strip extending over at least part of the pad backing and detachably attached to the disposable pad, and a longitudinally concertina folded disposal bag stored between the backing strip and the backing of the elongated pad bag wherein the disposal bag is unobtrusively in place when the disposable pad is in use and readily accessible for enclosing the disposable bag after use for hygienic disposal with the concertina fold being inverted lengthways allowing connection at one narrow end with minimal part of disposal bag remaining connected to allow maximising of bag volume.

The longitudinal concertina bag where concertina is inverted lengthways allows connection at one narrow end with minimal part of bag remaining connected to allow maximising of bag volume.

Still further the invention provides a disposable pad having an elongated absorbent pad element having a front side for contact with a body for receiving human excrements and an opposing rear side forming a pad backing, a backing strip extending over at least part of the pad backing and detachably attached to the disposable pad and a disposal bag mounted between the backing strip and the pad backing wherein the disposal bag is unobtrusively in place when the disposable pad is in use and readily accessible for enclosing the disposable bag after use for hygienic disposal the disposal bag being a folded in bag such that an inversion of the bag in operation reveals the fold which can then be further pulled out to provide a larger volume bag for enveloping the disposable pad.

The folded in bag prior to concertina provides the inversion of the bag in operation which reveals the fold which can then be further pulled out to provide a larger volume bag for enveloping the nappy.

The invention also provides a disposable pad having an elongated absorbent pad element, a backing strip and a disposal bag. The elongated absorbent pad element has a front side for contact with a body for receiving human excrements and an opposing rear side forming a pad backing. The backing strip extends over at least part of the pad backing and detachably attached to the disposable pad. The disposal bag mounted between the backing strip and the pad backing wherein the disposal bag is unobtrusively in place when the disposable pad is in use and readily accessible for enclosing the disposable bag after use for hygienic disposal. The backing strip extends along the length of the nappy over the disposal bag and where the backing strip forms part of the disposal bag and wherein the backing strip is longer than the disposal bag structure with the added length acting as a readily handled tag, covers the bag and stored products and provides a continuous outer surface to minimise ridges and enables activation of the bag without separate handling. The disposable pad further having a double action backing strip formed by a first part attached to the disposable pad forming a storage pouch holding a disposal bag and a second part separately attached to the disposable pad able to hold other hygienic products and wherein the storage pouch is opened by pulling on the backing strip to first open the second part and release the further hygienic products and by further pulling the bag structure is able to enclose the used disposable pad for hygienic disposal, wherein the attachment of the second part is more frangible than the attachment of the first part. The disposable pad further having a longitudinally concertina folded disposal bag stored between the backing strip and the backing of the elongated pad with the concertina fold being inverted lengthways allowing connection at one narrow end with minimal part of disposal bag remaining connected to allow maximising of bag volume wherein the folded in bag prior to concertina has an inversion of the bag in operation reveals the fold which can then be further pulled out to provide a larger volume bag for enveloping the disposable pad.

Overall it can be seen the invention allows a soiled nappy/diaper or sanitary/incontinence pad to be sealed in its own in-built bag after use. The system is designed to facilitate an easy, smell-free and hygienic disposal of a used nappy/diaper and sanitary/incontinence pad, this nappy/diaper or sanitary/incontinence pad makes it far more convenient to change a baby's nappy/diaper or when changing a sanitary/incontinence pad when travelling and on other occasions when it is not possible to change a baby in private or when disposal of the soiled nappy diaper or sanitary/incontinence pad may not be immediately possible. This is due to everything that is required to clean the baby or person is being contained within the unit, which after the bag being released and expanded, the nappy/diaper and sanitary/incontinence pad is then sealed in the bag and so packed and neutralised for disposal when convenient.

In one form of the invention there is a disposable nappy/diaper or sanitary/incontinence pad that contains a plastic bag with an inbuilt drawstring or tie-able plastic tags, which opens to wrap around and over the entire nappy/diaper and sanitary/incontinence pad.

The plastic bag is stored under two flaps at the bottom of the nappy. When the flaps are opened the bag is automatically released and envelope the entire nappy/diaper or sanitary/incontinence pad to enclose the soiled nappy/diaper or sanitary/incontinence pad. Also under the flaps that cover the bag are a number of pre-moistened baby wipes or towellettes. This facilitates the cleaning of the baby or person without the need for any additional baby wipes or the like. The bag itself is lightly scented and has odour neutralising properties.

In another form of the invention, the sealing system of the bag is performed by having two closure tabs at the free end of the bag which are integral to the sealing of the bag itself and that are approximately 50 mm by 25 mm in size. These tabs are tied together in order to seal the bag both from fluids and odours emanating from the bag.

In still another form of the invention the sealing system on the bag is performed by a simple drawstring mechanism at the free end of the bag in order to permanently seal the bag both from fluids and odours emanating from the bag.

In an even further form of the invention and in any one, all or any combination thereof of the forms of the invention previously mentioned, there may be an inclusion of a number of pre-moistened baby wipes or towellettes.

### BRIEF DESCRIPTION OF THE DRAWINGS

In order that the invention can be more readily understood various embodiments will be described by way of illustration only with reference to the drawings wherein.

FIG. 1 is an orthographic plan view of a first embodiment of a disposable pad and disposing method and apparatus;

FIG. 2 is an environmental orthographic view of the disposable pad of FIG. 1 showing a compartment flap;

FIG. 3 is an isometric cross sectional view taken along the lines of 2-2 of FIG. 1 with an expanded section FIG. 3a showing the position of the fixing points around Y;

FIG. 4 is an environmental orthographic view around point Y-Y showing the compartment flap open;

FIG. 5 is an isometric cross sectional view taken along the lines of 2-2 of FIG. 1 with compartment flap open;

FIG. 6 is an environmental orthographic side view of the disposable pad of FIG. 1 showing the compartment flap open and initiating of the enveloping process of the disposal bag;

FIG. 7 is an environmental orthographic view of the disposal pad of FIG. 1 showing the compartments open, in a second stage of enveloping process of the disposal bag;

FIG. 8 is an isometric cross sectional view taken along the lines of 2-2 of FIG. 1 with compartment flaps with disposal bag expanded being pulled downwards around pivot point X; and

FIGS. 9 and 10 are orthographic side views of the disposable pad showing the compartment open, extending and expanding disposal bag with the tie tabs being deployed;

FIG. 11 is an orthographic side view of the disposable pad showing the compartment disposal bag expanded around the nappy/diaper with the tie tabs being deployed;

FIG. 12 is a perspective view of a disposal bag in one form for use in an embodiment of the disposable pad and disposing method and apparatus of the invention;

FIG. 13 is a perspective view of the disposal bag of FIG. 12 in a first construction mode with inverted top portion;

FIG. 14 is an orthographic view of the disposal bag of FIGS. 12 and 13 in a second construction mode with concertina folding;

FIG. 15 is an orthographic cutaway view of the disposal bag of FIG. 14; FIG. 16 is an orthographic plan view of a second embodiment of a disposable pad and disposing method and apparatus;

FIG. 17 is an environmental orthographic view of the disposable pad of FIG. 16 showing a compartment flap;

FIG. 18 is an isometric cross sectional view the disposable pad of FIG. 16 taken along the lines of 3-3 of FIG. 16;

FIG. 19 is an environmental orthographic view the disposable pad of FIG. 16 showing the compartment flap open;

FIG. 20 is an isometric cross sectional view taken along the lines of 3-3 of FIG. 16 with compartment flap open;

FIG. 21 is an environmental orthographic side view of the disposable pad of FIG. 16 showing the compartment flap open and initiating of the enveloping process of the disposal bag;

FIG. 22 is an isometric cross sectional view taken along the lines of 3-3 of FIG. 16 with compartment flaps with disposal bag expanded being pulled downwards around pivot point;

FIG. 23 is an orthographic side views of the disposable pad of FIG. 16 showing the compartment open, extending and expanding disposal bag;

FIG. 24 is an isometric cross sectional view taken along the lines of 3-3 of FIG. 16 with compartment flaps with disposal bag expanded being pulled downwards and enclosing nappy/diaper;

FIG. 25 is an isometric cross sectional view of a third embodiment of a disposable pad and disposing method and apparatus in accordance with the invention with a view similar to disposable pad of FIG. 16 taken along the lines of 3-3 of FIG. 16;

FIG. 26 is an isometric cross sectional view of the disposable pad and disposing method and apparatus of the third embodiment of FIG. 25 with first compartment flap being pulled downwards and opened;

FIG. 27 is an isometric cross sectional view of the disposable pad of FIG. 25 showing the compartment flap open and initiating of the enveloping process of the disposal bag; and

FIG. 28 is an isometric cross sectional view taken of FIG. 25 with disposal bag expanded, pulled downwards and enclosing nappy/diaper.

### DETAILED DESCRIPTION OF THE PREFERRED EMBODIMENTS

Referring to the drawings, there is shown a disposable pad 6 which in this case is a disposable nappy also known as a disposable diaper. The nappy/diaper 6 has a substantially I-shape formed by two spaced rectangular end parts 6a, 6b separated by an elongated narrower substantially rectangular central portion 6c with outside edges 6d extending outside to the wider width of the end parts 6a, 6b. An elongated absorbent pad element extends along a front side of the nappy/diaper 6 across the end parts and the central portion 6c and particularly along a central rectangular strip 6e extending the entire length of the nappy/diaper for contact with a baby's body for receiving human excrements.

An opposing rear side of the central rectangular strip 6e of the nappy/diaper 6 has an exterior surface 15 forming a pad backing strip. Two elongated flaps 21, 22 extend over part of the exterior surface 15 and are detachably attached to the nappy/diaper 6. The first flap 21 is attached at one end near a first end part 6a of the nappy/diaper 6. The attachment is laterally across the central rectangular strip 6e to form a flap pivot point around Y-Y and extend to an end 8. The second flap 22 is attached at opposing end part 6b of the nappy/diaper 6 across the central strip 6e at X-X. The second flap 22 extends further than the first flap 21 such that the first flap 21 overlaps the second flap 22.

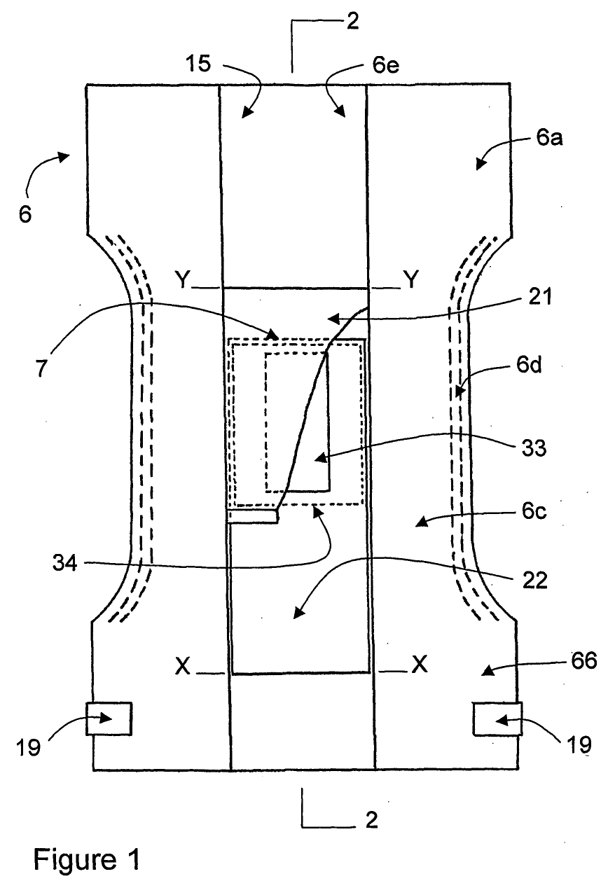

Aids for cleaning the baby's bottom such as towellettes 33 sold under the trademark Wet Wipes can be retained between the overlap of the first flap 21 and the second flap 22. A disposal bag 34 is mounted between the second flap 22 and the backing strip 15. In FIG. 1 there is shown the exterior surface 15 of the nappy/diaper 6 with compartment flap 21 pivot point Y-Y displayed and also compartment flap 22 pivot point X-X.

Also displayed in a partial cutaway section are the towellettes 33 and bag 34. It can be seen that the aids 33 and the disposal bag 34 are unobtrusively in place when the nappy/diaper 6 is in use and readily accessible for use and for enclosing the disposable bag after use for hygienic disposal.

In use the nappy/diaper 6 is fastened around a baby with the end parts 6a and 6b forming a circumferential waist band joined by adhesive tape 19 at sides of second end part 6b. The central portion 6c extends in U form under the baby with the curved outside edges 6d constricting around the legs of the baby to form a substantially enclosed structure with the absorbent pad able to absorb or hold excrement.

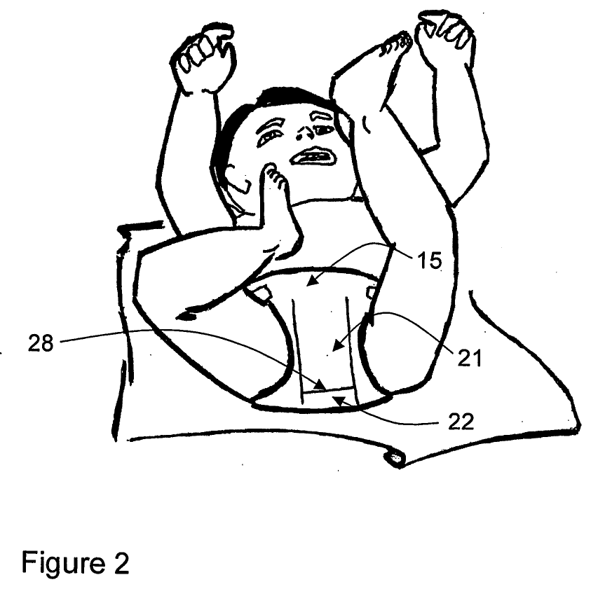

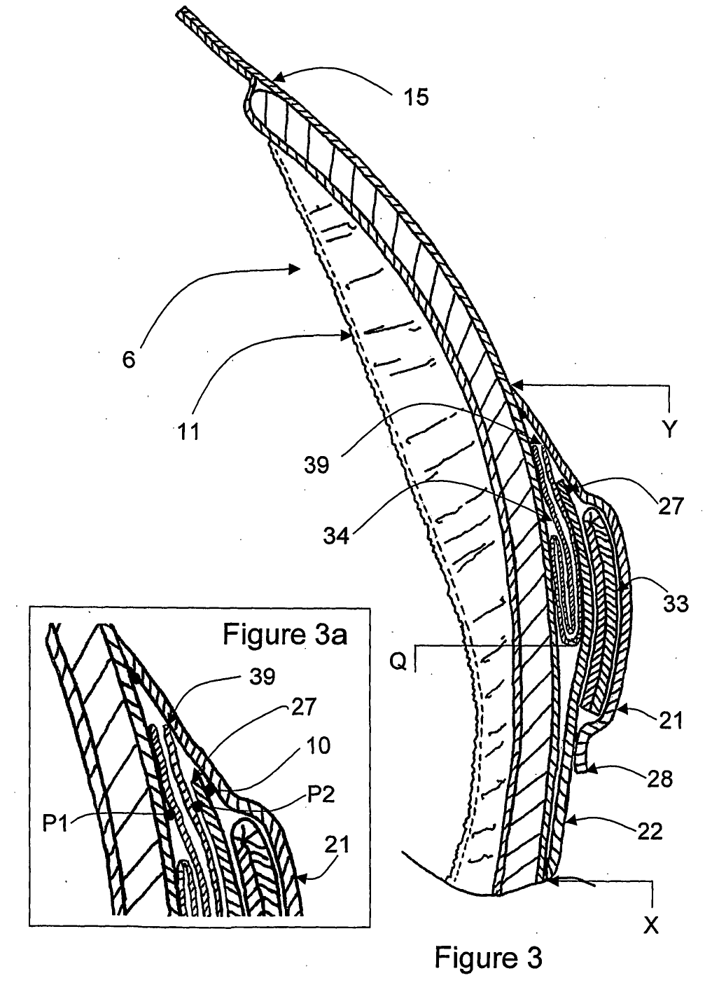

FIG. 2 shows nappy/diaper 6 in-situ and the compartment flap 21 and 22 closed with the bottom part of the elongated edge 28 of compartment flap 21. FIG. 3 is an isometric view taken along the lines of 2-2 of FIG. 1 with an expanded section FIG. 3a of FIG. 3 to highlight the position of the fixing points PI and P2 and the non-permanent fixing point between compartment flap 2's outside and end edge 7 and compartment flap 1's inner side. Also displayed is the free end 39 of the bag 34. Displayed in FIG. 3 is the inner side porous layer 11 of the nappy diaper 6 and bag 34 transverse folding pivot point Q.

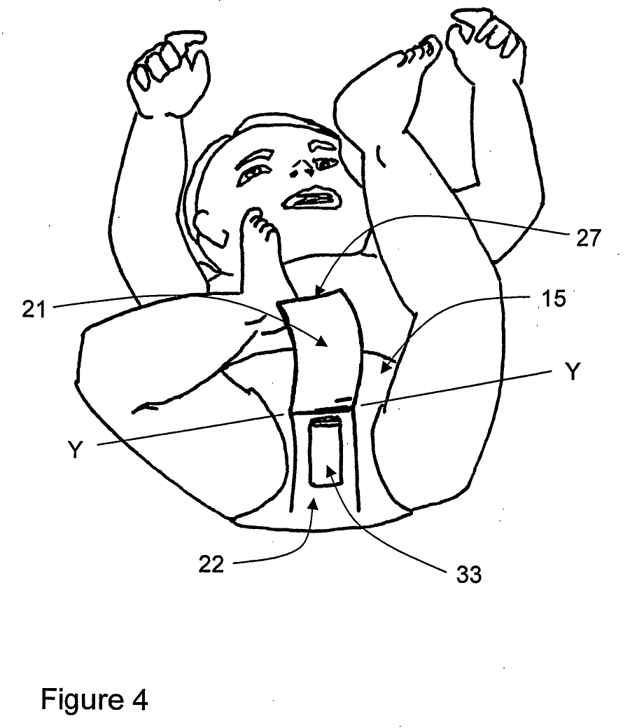

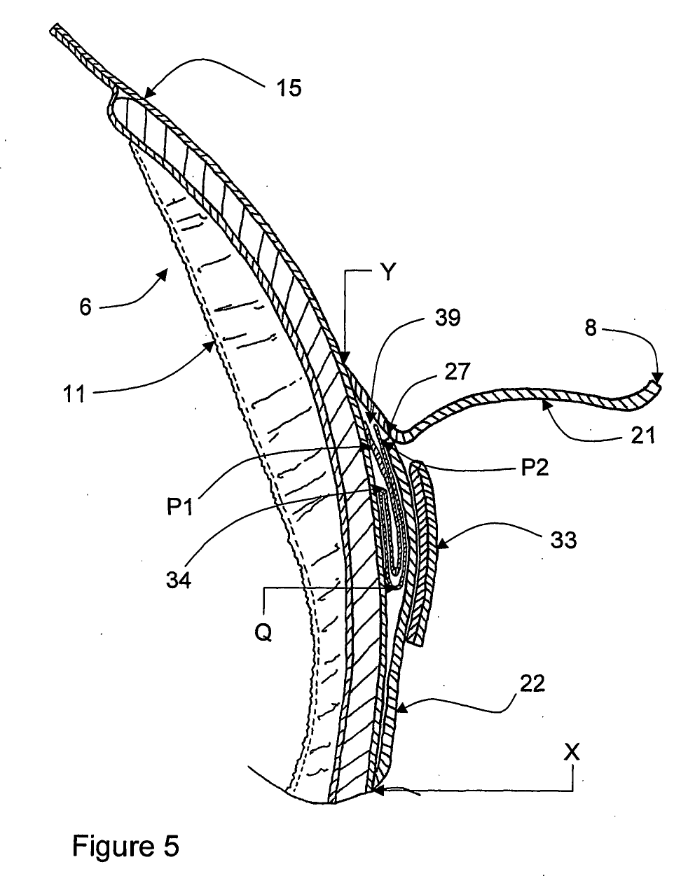

In FIGS. 4 and 5 there is shown the use of the nappy/diaper 6 of this embodiment in a first stage. At this stage the compartment flap 21 is open and pivoting around point Y-Y displaying the towellettes 3 and compartment flap 22 is still un-opened. FIG. 5 shows the process in cross sectional view with internal components being affected and displayed with compartment flap 21 open and the towellettes 33 accessible.

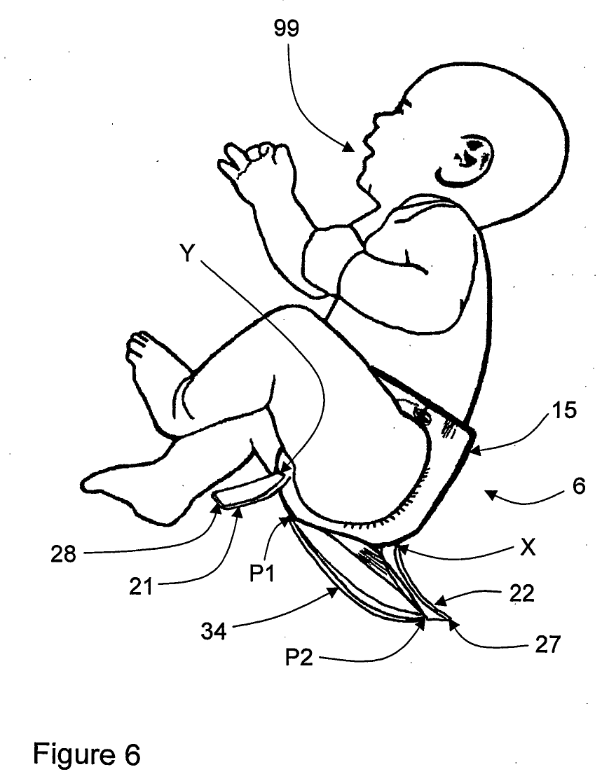

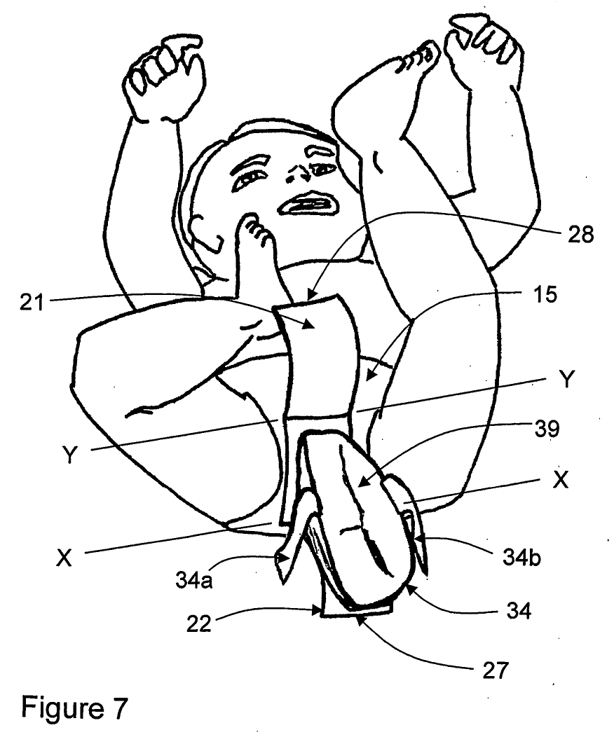

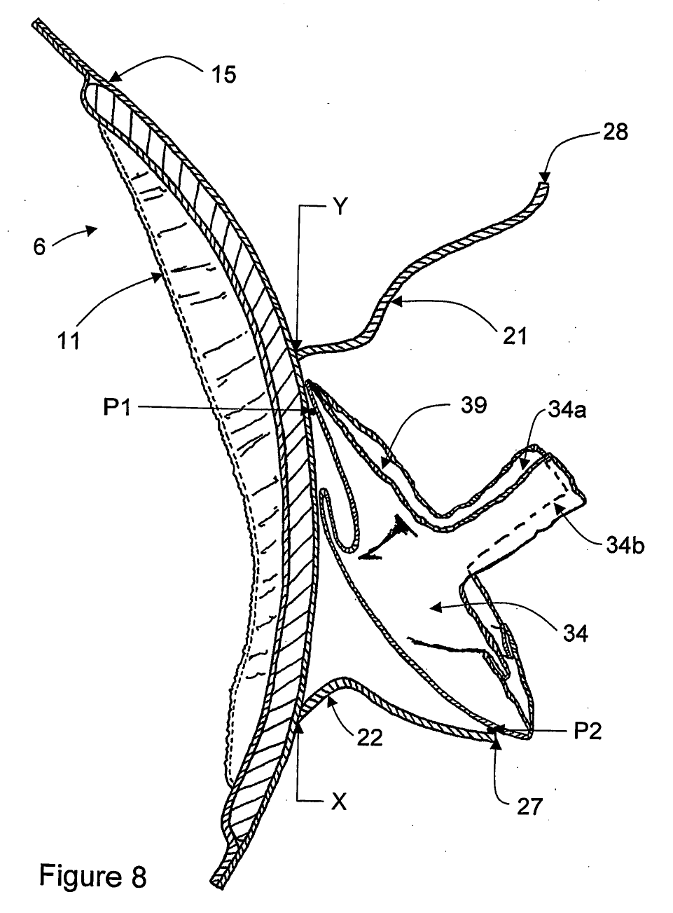

A further stage of the deployment of the bag 34 is shown in FIGS. 6 to 10. A first part of this stage is displayed in FIGS. 6, 7 and 8 showing the compartment flap 21 open pivoting around point Y and compartment flap 22 open pivoting around point X, extending and expanding bag 34 to initiate the enveloping process of the nappy/diaper 6. FIG. 7 clarifies the process further showing the compartment flap 21 open and compartment flap 22 open, extending and expanding bag 34 around pivot point X to initiate the enveloping process of the nappy/diaper 6 which includes tie tabs 34a and 34b extending from the open top of the bag 34 being released and made available for deployment. In FIG. 8 taken along the lines of 2-2 of FIG. 1 compartment flap 21 and 22 is open with bag 34 expansion initiated through the opening of compartment flap 22 and being pulled downwards around pivot point X and compartment flap 1 being fully extended and pivoting around point Y.

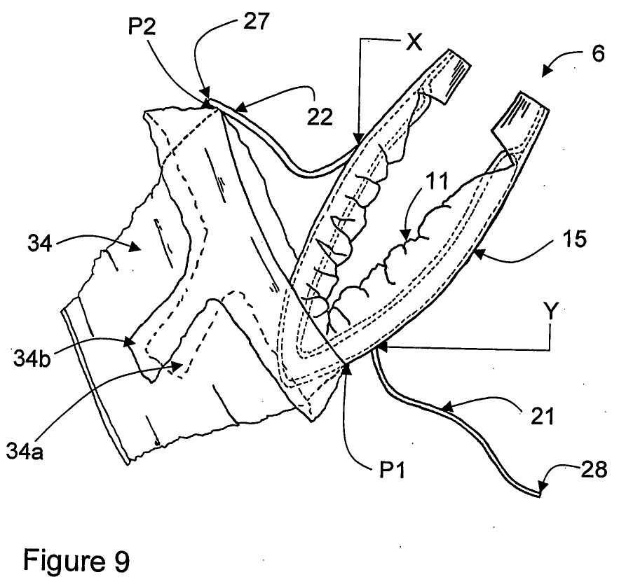

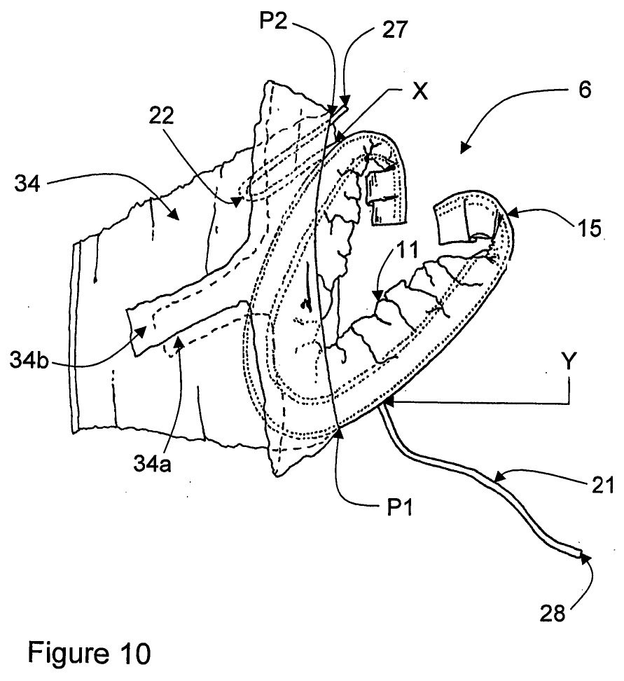

The second part of this stage of deployment of the bag 34 shown in FIGS. 9 and 10 includes a side cross sectional view of the nappy/diaper 6 showing the compartment flap 21 open and compartment flap 22 open, extending and expanding bag 34 around pivot point X to initiate the enveloping process of the partly folded nappy/diaper 6 with the tie tabs 34a and 34b being deployed. FIG. 10 shows the compartment flap 21 open and compartment flap 22 open, extending further and expanding bag 34 around pivot point X to continue the enveloping process of the partly folded nappy/diaper 6 with the tie tabs 34a and 34b being deployed and the bag 34 being half way around the nappy/diaper 6.

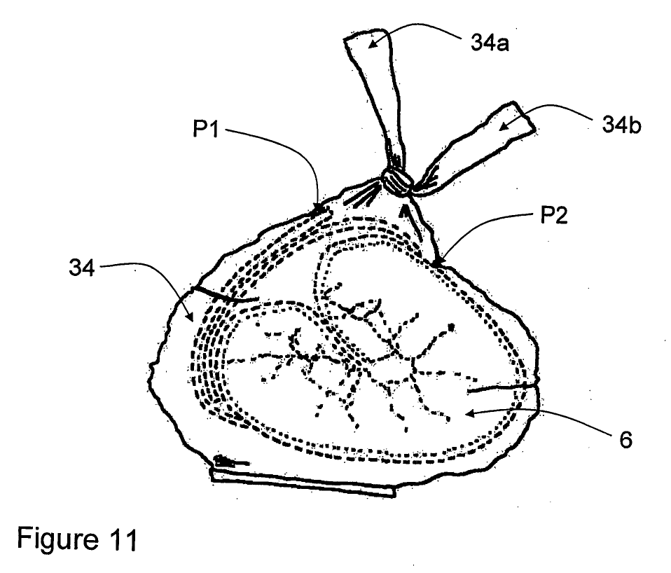

Lastly FIG. 11 has the nappy/diaper 6 completely enveloped by bag 34 and the tie tabs 34a and 34b having been tied and so enclosed permanently the bag 34 and its contents for ease and comfort of disposal. The bags 34 material whether plastic or otherwise can be separately coloured and constructed of one or more of a range of known manufacturing materials. The sealing device can vary as can the actual size of the bag 34.

The flexible permanent and non-permanent adhesives are non-toxic and may vary as to safety requirements, manufacturing processes, and marketing requirements.

The enclosure material, dimensions, and adhesive fixing point 27, P1 and P2 locations may vary as to safety requirements, manufacturing processes, and marketing requirements.

The plastic bag 34 fastening tabs 34a and 34b and the elongated compartment flaps 21 and 22, as well as the compartment fixing points p1 and P2 may vary in dimensions, colour, manufacturing material, and locale on the nappy/diaper or sanitary/incontinence pad 6 itself depending on the size of the nappy/diaper or sanitary/incontinence pad 6, safety requirements, manufacturing processes, and marketing requirements.

Thus what has been described above is a nappy/diaper or sanitary/incontinence pad 6 with a drawstring or otherwise 34a and 34b sealing plastic bag comprising moist baby wipes or towellettes 33 to conveniently dispose of the nappy/diaper or sanitary/incontinence pad 6 in a hygienic and convenient manner.

Disposable nappy and/or sanitary/incontinence pad 6 assembly incorporating wet baby wipes 33 and an integral bag 34 for enclosing and subsequent disposal of the soiled nappy 6 and baby wipes 33.

Therefore the nappy/diaper or sanitary/incontinence pad 6 assembly has an integral pouch 34 made of a flexible fluid impermeable material in an elongated shape aligned inline lengthways with the nappy/diaper or sanitary/incontinence pad 6 at the outside 15 bottom of the nappy/diaper or sanitary/incontinence pad 6, containing a number of wet baby wipes 33 or pre-moistened towellettes 33 in the primary compartment, and in the secondary compartment formed by the cavity created between the elongated compartment flap 22 and the impermeable outer layer 15 of the nappy/diaper or sanitary/incontinence pad 6 section, where a salable pre formed bag 34 is situated, that is fluid and odour impermeable, in order to enclose the nappy/diaper or sanitary/incontinence pad 6 therein.

The bag is fixed to the bottom P1 and P2 of the nappy/diaper or sanitary/incontinence pad 6 through utilization of a flexible adhesive to permanently fix the bag 34 to the nappy/diaper or sanitary/incontinence pad 6. Secondary compartment can only be opened subsequent to the opening of the primary compartment. The fluid impermeable outer layer of the secondary compartment and the fluid impermeable inner layer of the primary compartment form a sealed pocket through edging of flexible adhesive 10, where the pocket contains the pre moistened baby wipes or towellettes 33.

The nonporous bag 34 is packed in concertina fashion in a rectangular shape under the secondary flap 22 forming the secondary compartment. The bag 34 is fixed along P2 at the side of the free end 29 of the bag 34 with a permanent adhesive to the inner top edge 27 of the secondary compartment flap 22. This allows extraction of the bag 34 down and wrapping around the soiled nappy/diaper or sanitary/incontinence pad 6 as the secondary flap 22 is extended down and around the nappy/diaper or sanitary/incontinence pad 6.

The other side of the free end of the bag is fixed along P1 to the nappy/diaper or sanitary/incontinence pads 6 top end covered under elongated compartment flap 22. In one example fixing adhesive is applied 50 mm from the top of the free end 29 of the bag. The adhesive is of flexible permanent type that withstands the releasing of the flap 21 and flap 22 to extend and envelope the nappy/diaper or sanitary/incontinence pad 6 completely. The permanent flexible type adhesive is placed at the fixing points P2 inner top end of the secondary compartment flap and the top end of the cavity P1 made by the outer nappy wall 15 and the secondary compartment wall where the second fixing point is to the top end outer nappy wall P1 in the cavity.

The fluid and odour impermeable bag 34 comprises a sealing system 34a and 34b at the free end 9 of the bag 34 in order to permanently seal the bag 34 containing the soiled pre-moistened baby wipes or towellettes 33 and the nappy/diaper or sanitary/incontinence pad 6.

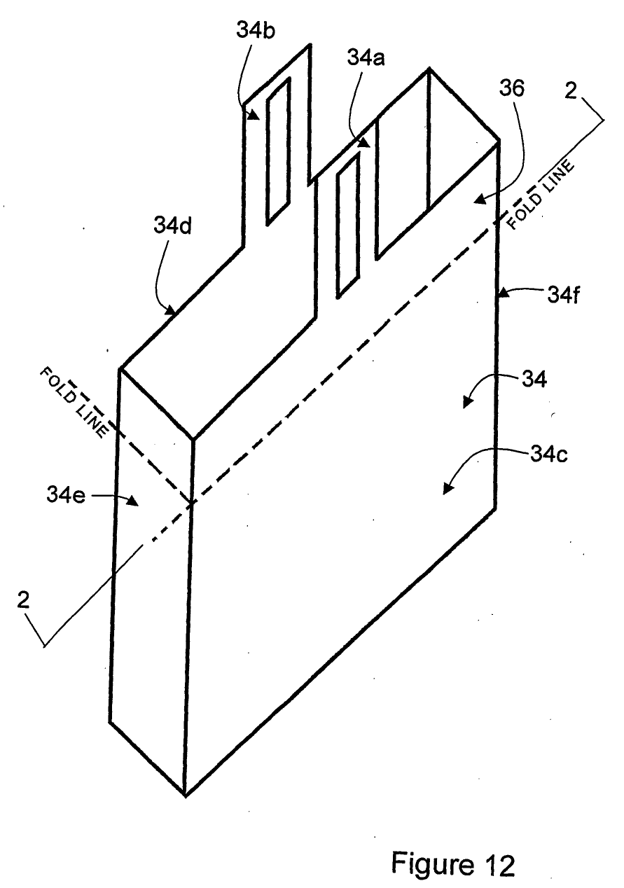

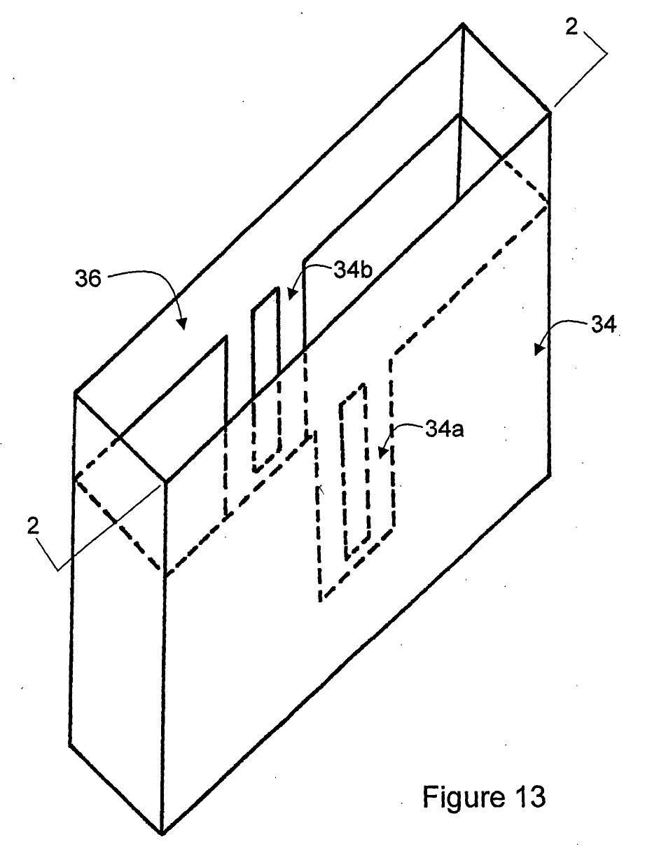

Referring to FIG. 12 to 15 there is shown the disposal bag 34 usable in embodiments of the invention wherein the disposal bag is substantially an open rectangular box shape having two narrow end panels 34e and 34f extending between two side panels 34c and 34d. A top portion of the disposal bag 36 leads to the open top and tie tabs 34a and 34b. This top portion 36 together with the tie tabs 34a and 34b can be inverted into the bag 34 to form an apparently smaller dimensioned bag 34. Clearly this disposal bag retains the same larger dimensions than the apparently smaller dimension bag 34 and the full volume can be used by reversing the inversion of the top portion 36 and tie tabs 34a and 34b.

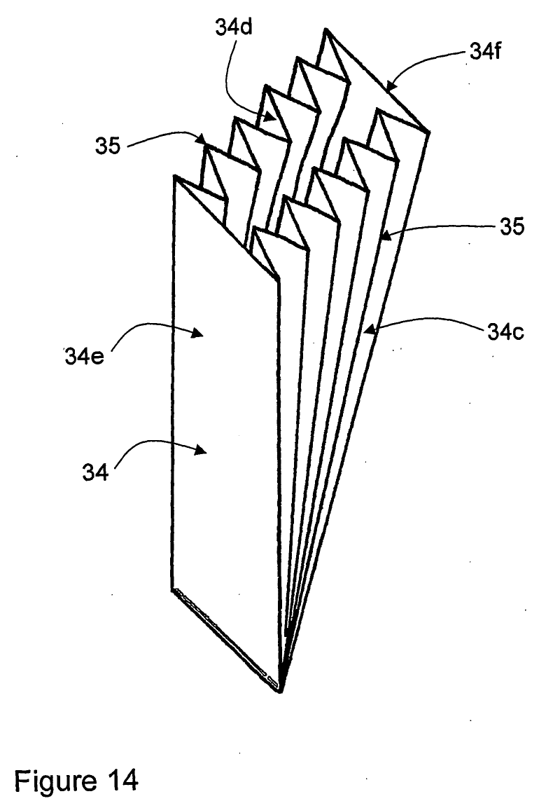

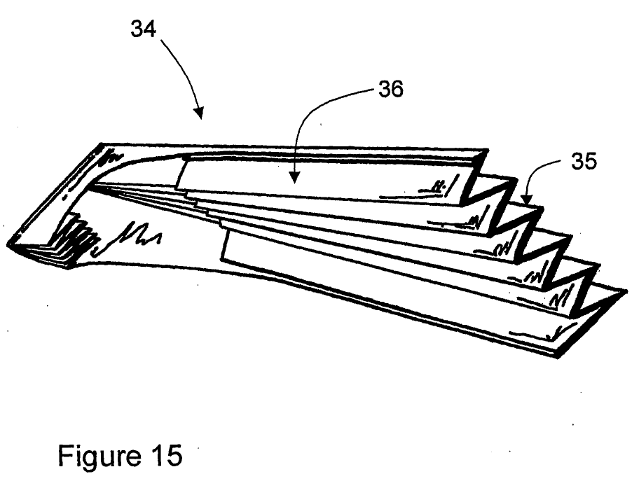

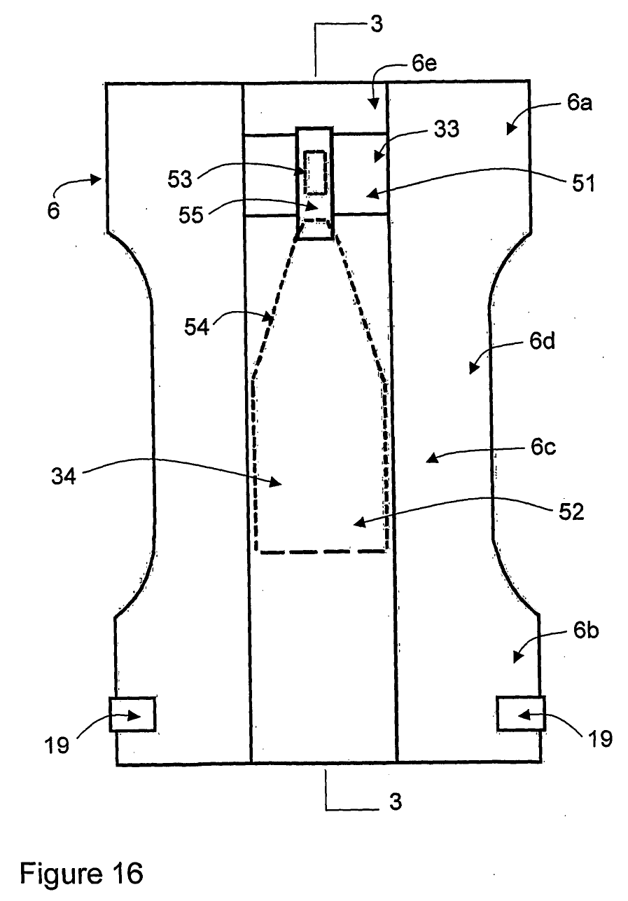

As shown in FIGS. 14 and 15 the apparently smaller dimension bag 34 can then be concertina folded along its longer dimensions in a symmetrical manner such that the end panels 34e and 34f become the greatest external dimension and the side panels 34c and 34d compress to minimal thickness. The concertina folds 35 retain and open top and preferably extend to the middle of the folded structure to minimise layers of folds and maximise volume of bag 34 for size of final concertina folded disposal bag 34.

In use therefore the concertina folded bag 34 attached to the backing 15 of the disposable pad is able to unfold and then have its top portion 36 and tie tabs 34a and 34b extracted from the open top of the bag 34. By further inversion the apparently enlarged concertina folded disposal bag 34 is able to envelope the used disposable pad.

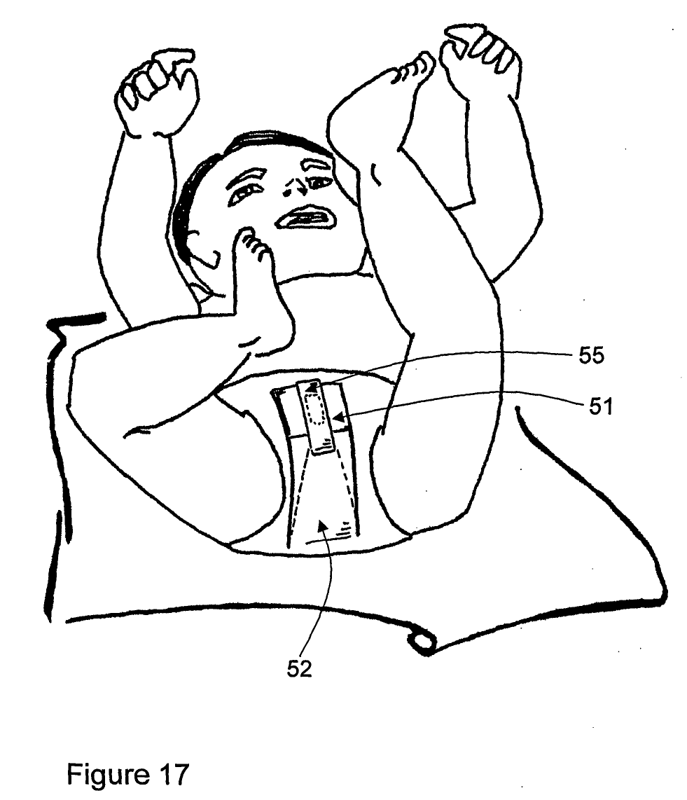

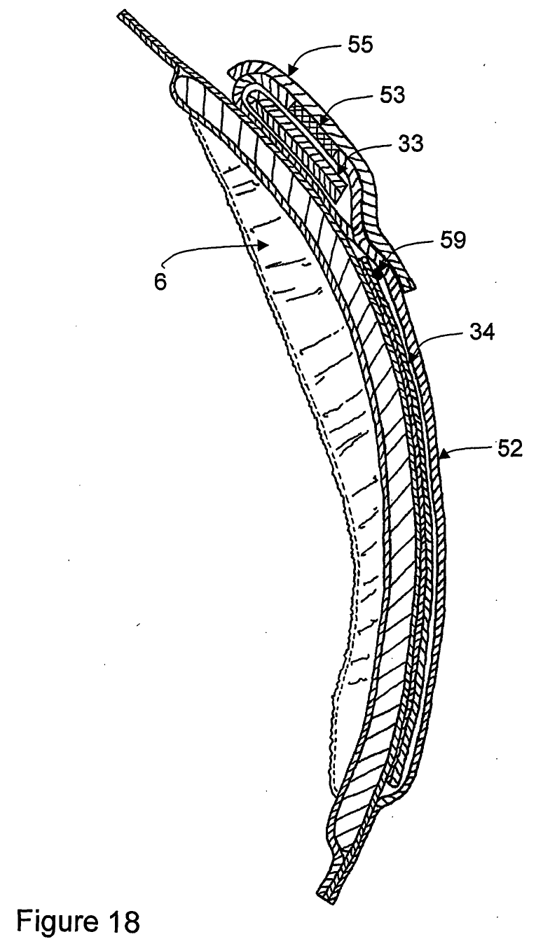

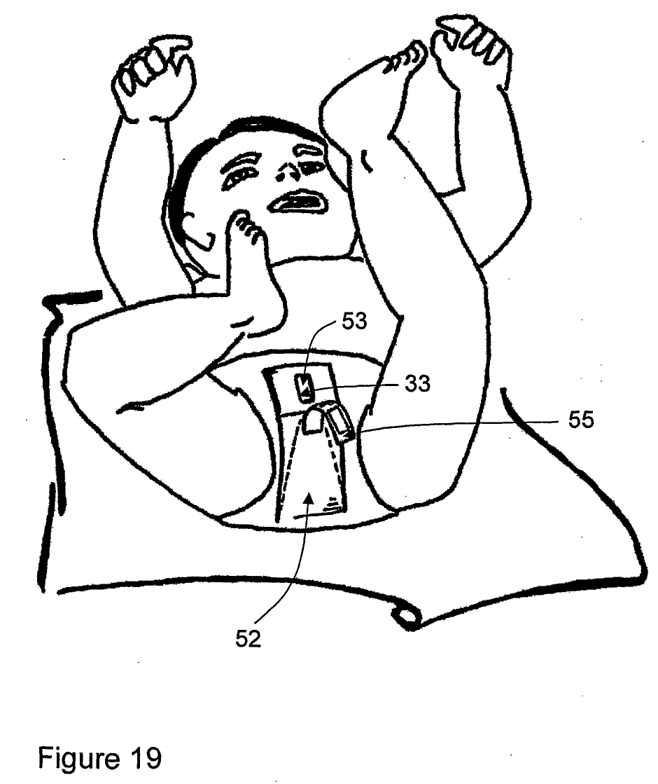

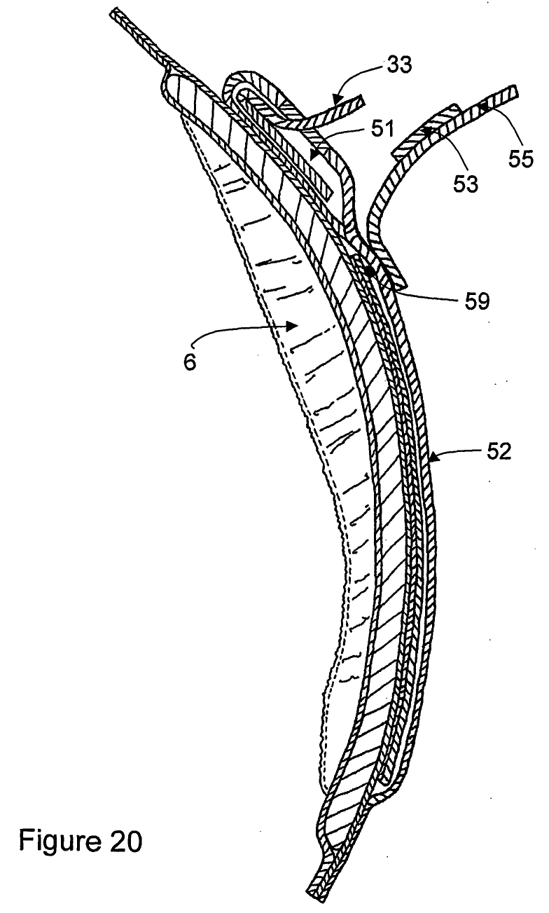

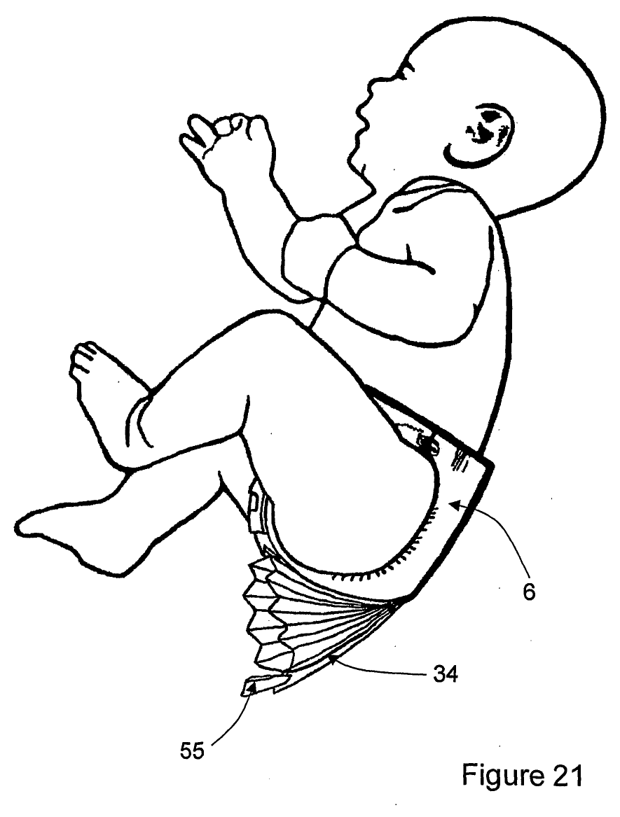

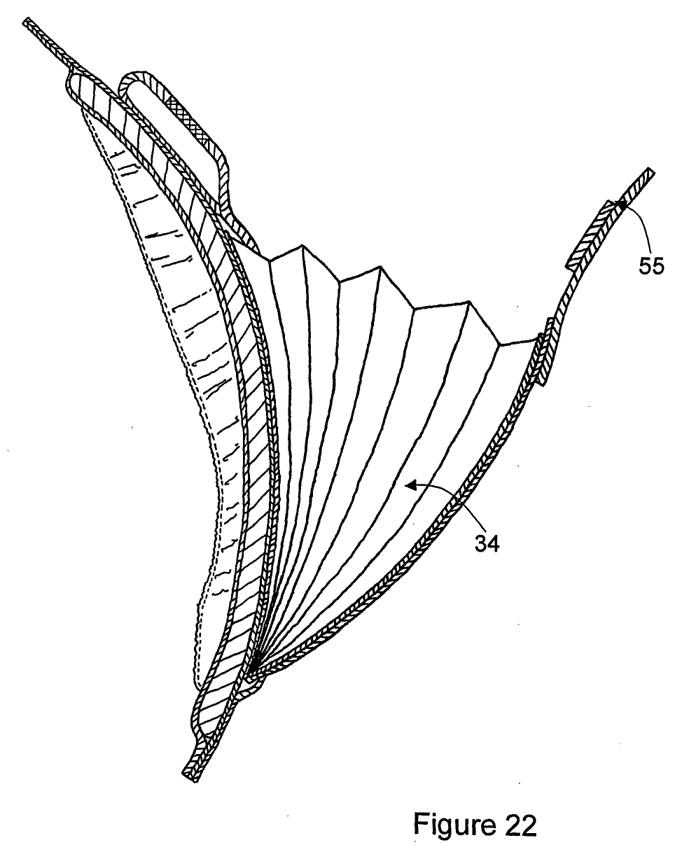

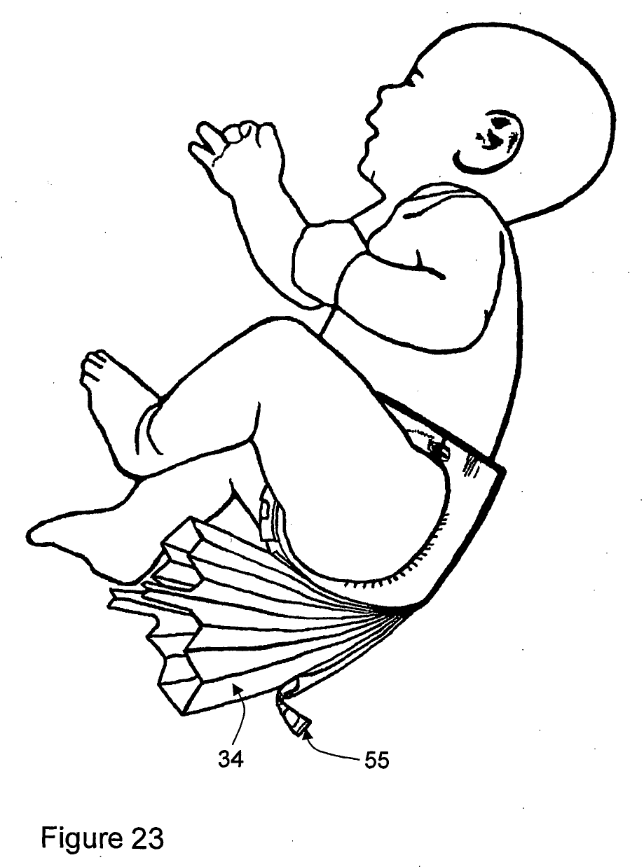

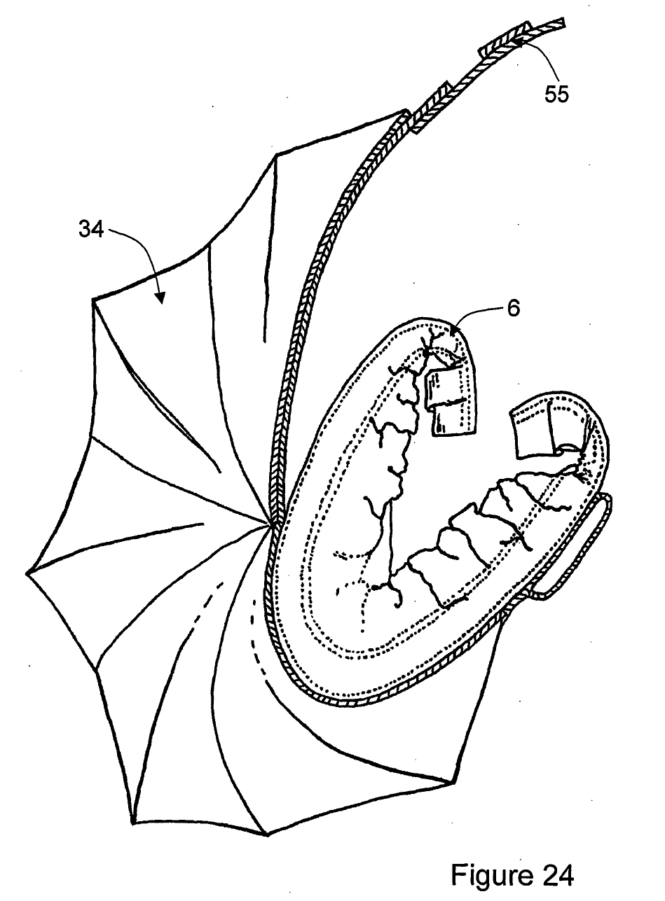

Referring to FIGS. 16 to 24 there is shown a second embodiment of the disposable pad of the invention where like numbers refer to like structures of the first embodiment. In this embodiment in particular there is the forming of a disposable pad according having a double action backing strip formed by a first part attached to the disposable pad forming a storage pouch to hold hygienic products and a second part separately attached to the disposable pad holding a disposal bag and wherein the storage pouch is opened by pulling on the backing strip to open the first part and release the hygienic products and by further pulling open the second part releasing the disposal bag for enclosing the used disposable pad for hygienic disposal.

The attachment of the first part is more frangible than the attachment of the second part to prevent accidental opening of the second part.

The first part includes a substantially non porous sealing panel having a frangible line covered by a sealing means to form a sealed pouch able to hold hygienic products in a substantially sealed form, wherein the sealing means can be removed to break the frangible line to open the sealed pouch for access to the hygienic products.

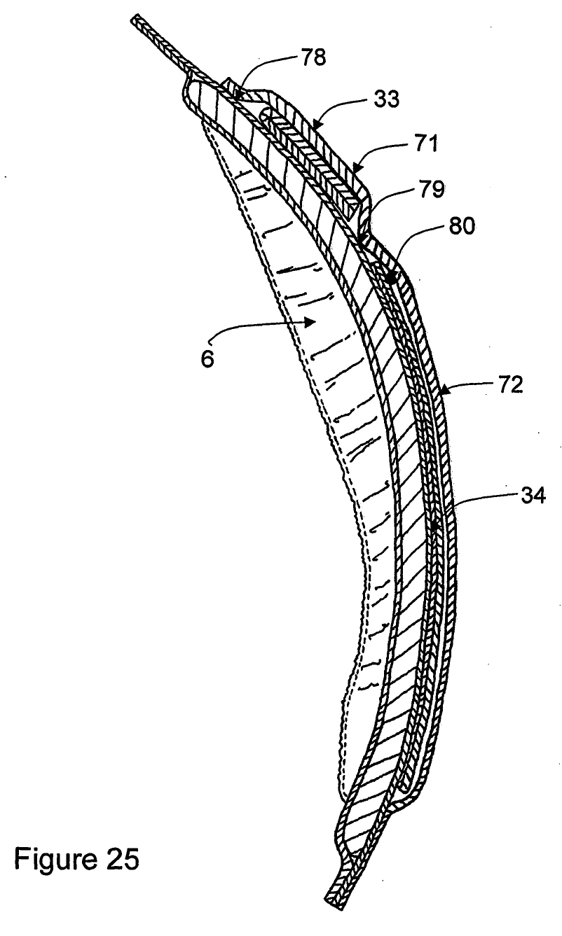

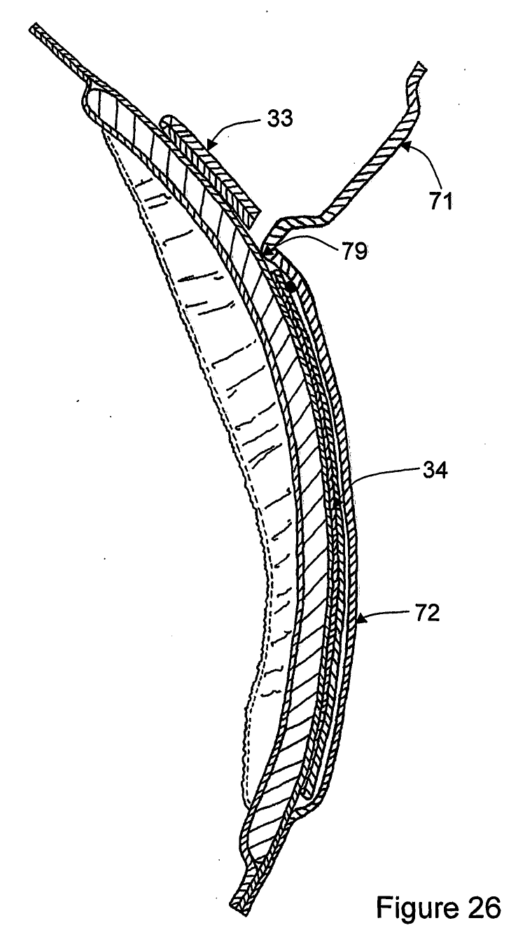

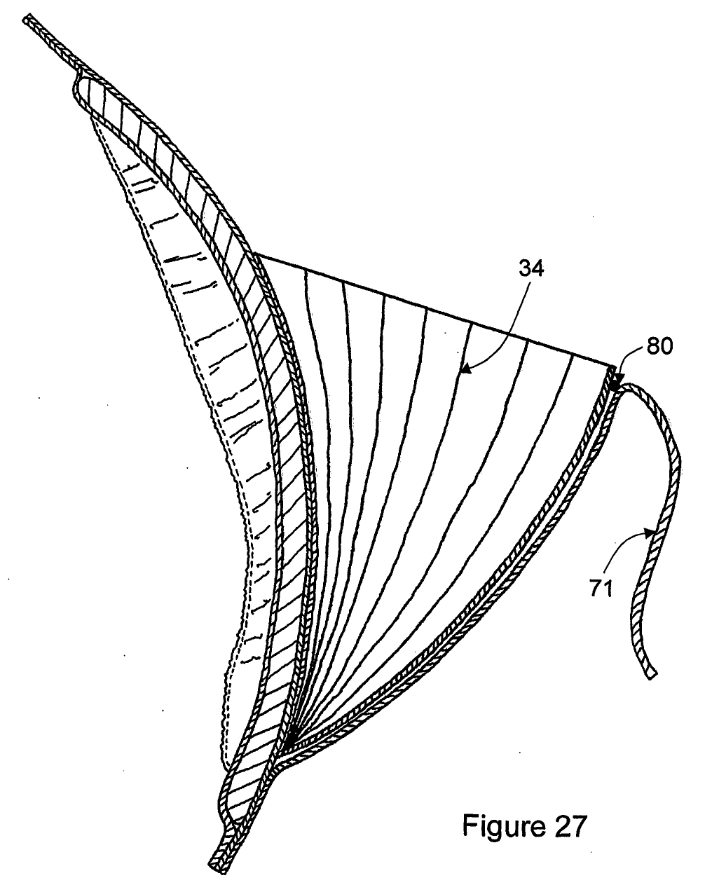

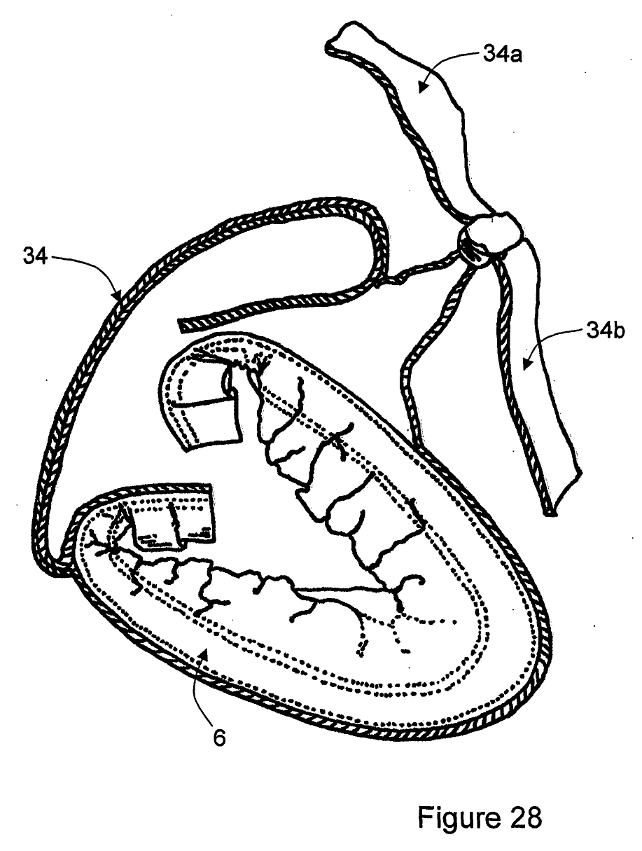

Referring to FIGS. 25 to 28 there is shown a third embodiment of the invention substantially similar to the second embodiment and including first and second pouches opened by a single tab. However unlike the second embodiment in this variation of the invention the first pouch is not hermetically sealed.

It should be understood that the foregoing relates to only a limited number of preferred embodiments of the invention which have been shown by way of example only, and that it is intended to cover all changes and modifications of the examples of the invention herein chosen for the purpose of the disclosure, which do not constitute departures from the spirit and scope of the invention as defined by the following claims.

1-20. (canceled)

21. A disposable pad having:
an elongated absorbent pad element having a front side for contact with a body for receiving human excrements and an opposing rear side forming a pad backing;
and having a double action backing strip extending over at least part of the pad backing and detachably attached to the disposable pad;
and with a disposal bag mounted between the backing strip and the pad backing wherein the disposal bag is unobtrusively in place when the disposable pad is in use and readily accessible for enclosing the disposable bag after use for hygienic disposal;
and the backing strip formed by a first part attached to the disposable pad forming a storage pouch to hold hygienic products and a second part separately attached to the disposable pad holding a disposal bag and wherein the storage pouch is opened by pulling on the backing strip to open the first part and release the hygienic products and by further pulling open the second part releasing the disposal bag for enclosing the used disposable pad for hygienic disposal.

22. A disposable pad according to claim 21 wherein the attachment of the first part is more frangible than the attachment of the second part to prevent accidental opening of the second part.

23. A disposable pad according to claim 22 wherein the pad is a sanitary pad.

24. A disposable pad according to claim 22 wherein the pad is a disposable nappy or diaper having a substantially “I” shape with a central elongated absorbent pad and cross bands at each end forming a waist band with attachment means to retain the disposable nappy or diaper on the baby or infant; and
wherein the disposal bag is sized to fit the disposable nappy or diaper after use.

25. A disposable pad according to claim 24 wherein the backing strip extends along the length of the nappy over the disposal bag and where the backing strip forms part of the disposal bag.

26. A disposable pad according to claim 24 wherein the backing strip is longer than the disposal bag structure with the added length acting as a readily handled tag.

27. A disposable pad according to claim 24 wherein the backing strip covers the bag and stored products.

28. A disposable pad according to claim 27 wherein the backing strip provides a continuous outer surface to minimize ridges and enables activation of the bag without separate handling.

29. A disposable pad according to claim 22 wherein the first part includes a substantially non porous sealing panel having a frangible line covered by a sealing means to form a sealed pouch able to hold hygienic products in a substantially sealed form, wherein the sealing means can be removed to break the frangible line to open the sealed pouch for access to the hygienic products.

30. A disposable pad according to claim 29 and having a concertina folded disposal bag stored between the backing strip and the backing of the elongated pad with the concertina folded disposal bag having a height greater than its width in concertina folded mode and being positioned on the pad lengthways to allow maximizing of bag volume.

31. A disposable pad according to claim 30 wherein at least a portion of the concertina folded disposal bag remains attached to the pad such that the disposal bag is inverted around the used disposable pad.

32. A disposable pad according to claim 31 wherein the concertina folded disposal bag has an inversion of the top of the bag which in use can then be further pulled out to provide a larger volume bag for enveloping the disposable pad.

33. A disposable pad having:
an elongated absorbent pad element having a front side for contact with a body for receiving human excrements and an opposing rear side forming a pad backing;
a backing strip extending over at least part of the pad backing and detachably attached to the disposable pad;
a disposal bag mounted between the backing strip and the pad backing wherein the disposal bag is unobtrusively in place when the disposable pad is in use and readily accessible for enclosing the disposable bag after use for hygienic disposal
wherein the backing strip extends along the length of the nappy over the disposal bag and where the backing strip forms part of the disposal bag and wherein the backing strip is longer than the disposal bag structure with the added length acting as a readily handled tag, covers the bag and stored products and provides a continuous outer surface to minimize ridges and enables activation of the bag without separate handling;
the disposable pad further having:
a double action backing strip formed by a first part attached to the disposable pad forming a storage pouch holding a disposal bag and a second part separately attached to the disposable pad able to hold other hygienic products and wherein the storage pouch is opened by pulling on the backing strip to first open the second part and release the further hygienic products and by further pulling the bag structure is able to enclose the used disposable pad for hygienic disposal, wherein the attachment of the second part is more frangible than the attachment of the first part; and having
a longitudinally concertina folded disposal bag stored between the backing strip and the backing of the elongated pad with the concertina fold being inverted lengthways allowing connection at one narrow end with minimal part of disposal bag remaining connected to allow maximizing of bag volume.
wherein the folded in bag prior to concertina has an inversion of the bag in operation reveals the fold which can then be further pulled out to provide a larger volume bag for enveloping the disposable pad.

34. A disposable pad, according to claim 33 wherein the pad is a sanitary pad.

35. A disposable pad according to claim 33 wherein the pad is a disposable nappy or diaper having a substantially “I” shape with a central elongated absorbent pad and cross bands at each end forming a waist band with attachment means to retain the disposable nappy or diaper on the baby or infant; and wherein the disposal bag is sized to fit the disposable nappy or diaper after use.

36. A disposable pad having:
an elongated absorbent pad element having a front side for contact with a body and an opposing rear side forming a pad backing;
a backing strip extending over at least part of the pad backing and detachably attached thereto, the backing strip forming at least part of an outer surface of the disposable pad and having an extended unattached portion forming a tag;
a disposal bag mounted in a folded form between the backing strip and the pad backing and having at least a portion attached to the pad backing and the disposal bag being inwardly folded within itself to provide a larger volume bag when unfolded;
wherein the disposal bag is unobtrusively in place allowing for normal use of the disposable pad and after use the disposal bag being readily accessible by pulling the tag to release the bag and by unfolding of the bag and inversion of the bag relative to the disposable pad enclose the disposable pad for hygienic disposal.

37. A disposable pad having:
an elongated absorbent pad element having a front side for contact with a body for receiving human excrements and an opposing rear side forming a pad backing;
a backing strip extending over at least part of the pad backing and at least partially detachably attached to the disposable pad;
a disposal bag mounted on the pad backing and released by the backing strip being at least partially detached the disposal bag having an enlargening opening when released wherein the disposal bag is unobtrusively in place when the disposable pad is in use and readily accessible and the opening enlarged for enveloping the disposable pad after use for hygienic disposal
and wherein the backing strip is a double action backing strip formed by a first part of the backing strip attached to the disposable pad forming a sealed storage pouch to hold hygienic products hermetically and a second part of the same backing strip separately attached to the disposable pad and holding a disposal bag, wherein the storage pouch is opened by pulling on the backing strip to open the first part and release the hygienic products and by further pulling the backing strip open the second part releases the disposal bag for enclosing the used disposable pad for hygienic disposal.

38. A disposable pad according to claim 37 wherein the attachment of the first part is more frangible than the attachment of the second part to prevent accidental opening of the second part.

39. A disposable pad according to claim 38 wherein the backing strip extends along the length of the disposable pad and forms an elongated side part of the disposal bag.

40. A disposable pad having:
an elongated absorbent pad element having a front side for contact with a body for receiving human excrements and an opposing rear side forming a pad backing;
a double action backing strip formed by a first part attached to the disposable pad forming a storage pouch to hold hygienic products and a second part attached to the disposable pad to hold a disposal bag and wherein the storage pouch is opened by pulling on the backing strip to open the first part and release the hygienic products and by further pulling on the backing strip open the second part releasing the disposal bag for enclosing the used disposable pad for hygienic disposal;
the disposal bag mounted on the pad backing and having longitudinally concertina folds wherein the disposal bag is unobtrusively in place when the disposable pad is in use and releasable by the backing strip being at least partially detached, the disposal bag having an enlargening opening when released for enclosing the disposable pad after use for hygienic disposal.

41. A disposable pad according to claim 40 wherein the attachment of the first part is more frangible than the attachment of the second part to prevent accidental opening of the second part.

42. A disposable pad according to claim 41 wherein the backing strip extends along the length of the disposable pad and forms an elongated side part of the disposal bag.

43. A disposable pad according to claim 40 wherein the concertina folded disposal bag has an inwardly folded top of the bag which in use can then be further pulled out to provide a larger volume bag for enveloping the disposable pad.
